(19) United States
(12) Patent Application Publication
(10) Pub. No.: US 2015/0167029 A1
(43) Pub. Date: Jun. 18, 2015

(54) Method for Producing Isopentane Derivatives

(71) Applicant: Oxea GmbH, OBerhausen (DE)

(72) Inventors: Jens Klabunde, Dusseldorf (DE); Heinz Strutz, Moers (DE); Kristina Kockrick, Dusseldorf (DE)

(73) Assignee: Oxea GmbH, Oberhausen (DE)

(21) Appl. No.: 14/406,910

(22) PCT Filed: Jul. 1, 2013
(86) PCT No.: PCT/EP2013/063786

§ 371 Date: Dec. 10, 2014

(30) Foreign Application Priority Data
Jul. 2, 2012 (DE) 10 2012 105 878.4

(51) Int. Cl. C12P 7/52; C12P 7/24

(52) U.S. Cl. CPC C12P 7/52; C12P 7/24

(57) ABSTRACT

The present invention relates to a method of producing isopentane derivatives from fermentatively produced isobutene, the higher purity of which improves the method and the properties of the produced isopentane derivatives.

### CLAIM FOR PRIORITY

This application is a national phase application of PCT/EP2013/063786 FILED Jul. 1, 2013 which was based on application DE 10 2012 105 878.4 FILED Jul. 2, 2012. The priorities of PCT/EP2013/063786 and DE 10 2012 105 878.4 are hereby claimed and their disclosures incorporated herein by reference.

### TECHNICAL FIELD

The present invention relates to a method of producing isopentane derivatives particularly isovaleraldehyde (3-methylbutanal), pivalic acid, 3-methylbutanol, 3-methyl butyric acid, 2,3-dimethyl-2-butene, 2,3-dimethylbutane-2,3-diol (pinacol) and methyl-tert-butyl ketone (pinacolone) preferably from sources of renewable raw materials.

### BACKGROUND

Isopentane derivatives are important industrial products. Methods of producing e.g. isovaleraldehyde have long been known and are described inter alia in Ullmann's Encyclopedia of Industrial Chemistry, Wiley-VCH, 6th edition, 2003, volume 2, pages 73-74, and in W. J. Scheidmeir, Chem. Ztg. 96, 1972, Pages 383-387. Usually one starts from isobutene, which e.g. is extended by one carbon atom in an oxo or hydroformylation reaction. Because of the immense importance of such isopentane derivatives for the technical chemistry, however, it is constantly searched for further improvements with respect to alternative methods and alternative sources of raw materials for the production of isopentane derivatives.

The use of renewable raw materials as starting materials for the production of organic chemicals on an industrial scale is becoming increasingly important. On the one hand the resources based on petroleum, natural gas and coal should be conserved and on the other hand with renewable raw materials carbon dioxide is bound in an industrially useable carbon source, which in principal is inexpensive and available in large quantities. Examples for the use of renewable raw materials for the industrial production of organic chemicals include the production of citric acid, 1,3-propanediol, L-lysine, succinic acid, lactic acid, and itaconic acid.

Renewable raw materials are not yet used for the production of isopentane derivatives. Thus, the task will be to provide an alternative improved method for the production of isopentane derivatives preferably from sources of renewable raw materials. Herein it is of particular importance with regard to the use of isopentane derivatives that preferably isomer-free isobutene is used for the production of isopentane derivatives.

The term “isopentane derivatives” means in particular isovaleraldehyde (3-methyl butanal), pivalic acid and their esters, 3-methylbutanol, 3-methyl butyric acid and their esters, 2,3-dimethyl-2-butene, 2,3-dimethylbutane-2,3-diol (pinacol) and methyl-tert-butyl ketone (pinacolone) and mixtures of these compounds.

### SUMMARY OF INVENTION

The objective of providing an alternative, improved method for the production of isopentane derivatives is achieved by a method of producing isopentane derivatives comprising the steps of:
a) fermentative preparation of isobutene;
d) extension by one carbon atom in order to obtain an isopentane derivative; and
e) optionally further derivatisations.

It surprisingly has been found that the subsequent extension delivers the isopentane derivative with a high purity which in optionally subsequent derivatisations also increases the purity and yield. In the prior art methods are known in which isobutene is formed biochemically in high purity on a laboratory scale. Thus, however starting from the direct precursor 3-hydroxy-isovaleriate (3-hydroxy-3-methylbutyrate), Gogerty, D. S. and Bobik, T. A., 2010, Applied and Environmental Microbiology, pages 8004-8010, investigated the fermentative-enzymatic synthesis of isobutene, wherein according to GC no significant amounts of n-butene isomers were revealed in the valuable product.

The by-product carbon dioxide formed during the fermentation and optionally other inert gases may optionally be removed by suitable separation techniques in a conventional manner.

The further processing of the high-purity isobutene obtained by the fermentative process into the intermediates isovaleraldehyde and pivalic acid and optionally further derivatives due to the high selectivity to isobutene as C4-olefin in the fermentation product means a significant simplification of the process sequence into isovaleraldehyde and pivalic acid and corresponding derivatives.

According to a preferred embodiment of the invention steps a) and b) no purification of the isobutene to remove linear butene isomers is carried out between steps a) and b). In this embodiment of the invention the fementative method of the invention uses the high selectivity to isobutene as the C₄-olefin. Herein, “purification” means in particular (but not limited to) the following methods:
Distillation processes (which, however, are complicated by the fact that the separation of linear butene isomers occurring in the overall process requires a lot of effort, since the boiling points of the isomers are very close to each other, see Kirk-Othmer, Encyclopedia of Chemical Technology, 3rd edition, 1978, vol. 4, John Wiley & Sons Inc., pp. 358-360).
Purification or separation methods in which isobutene is separated due to the increased chemical reactivity by means of a chemical reaction, and then is converted back into isobutene. This includes methods such as reversible proton-catalyzed water addition to tert-butanol or the methanol addition to methyl-tert-butylether (see EP1489062). From these adducts then isobutene is recovered by a reverse reaction (see Weissermel, Arpe, Industrielle Organische Chemie, VCH Verlagsgesellschaft, 3rd edition, 1988, pp. 74-79).
Purification or separation methods in which isobutene is separated from linear butene isomers due to the more compact spatial molecular structure by means of suitable physical size exclusion methods, for example, by means of molecular sieves having an appropriate pore size, (see WO 2012040859, Weissermel, Arpe, Industrielle Organische Chemie, VCH Verlagsgesellschaft, 3rd edition, 1988, p. 74).

### DETAILED DESCRIPTION

The term “fermentative production” of isobutene means particularly that isobutene is derived either
by means of microorganisms, preferably from renewable raw materials; and/or
by a cell-free enzymatic method, also preferably from renewable raw materials.

Isobutene is—as far as is known—not a natural product in the sense that it is formed in metabolic processes in organisms in such amounts that an industrial use seems appropriate. However, isobutene is produced in very small amounts from naturally occurring microorganisms (U.S. Pat. No. 4,698,304; Fukuda, H. et al., 1984, From Agricultural and Biological Chemistry (1984), 48(6), pp. 1679-82). Thus, in the previously known embodiments of the invention, the fermentative preparation of isobutene is carried out by means of modified, non-natural microorganisms and the corresponding modified enzymes, respectively. Such microorganisms are known from US 2011165644 (A1), wherein in Example 13 the synthesis of isobutene from glucose in suitable microorganisms is discussed. In WO 2012052427 and WO 2011032934 further enzymatic reactions are described, which describe the formation of isobutene as a series of sequential enzymatic syntheses of
I) acetone into 3-hydroxyisovaleriate; and
II) 3-hydroxyisovaleriate into isobutene and carbon dioxide.

The enzymatically catalyzed decomposition of 3-hydroxyisovaleriate into isobutene and carbon dioxide is also discussed in Gogerty, D. S. and Bobik, T. A., 2010, Applied and Environmental Microbiology, pages 8004-8010. Here, according to GC, no significant amounts of n-butene isomers were revealed in the valuable product. Even in aqueous, non-enzymatically catalyzed systems one observes a spontaneous separation of carbon dioxide from 3-hydroxyisovaleriate under formation of isobutene, which further reacts with the present water in a balance reaction into tert-butanol (Pressman, D. and Lucas, H. J., 1940, Journal of the American Chemical Society, pages 2069-2081).

If this sequence of enzymatic syntheses described in I and II is included in a suitable microbial host organism which is capable of synthesizing acetone from metabolic precursors or to transport externally supplied acetone by means of a passive or active transport through the cell wall into the cell, by means of a non-natural microorganism derived in such a manner isobutene can be produced by a fermentative process with a good yield. Microorganisms that synthesize acetone from different carbohydrates have long been known and are described inter alia in Jones, T. D. and Woods, D. R., 1986, Microb. Reviews, pages 484-524. Taylor, D. G. et al., 1980, Journal of General Microbiology, 118, pages 159-170, describe microorganisms that use acetone as a sole carbon source and, thus, are able to transport acetone across the cell wall into the cell.

Another possible metabolic pathway proceeds via the reaction sequence:
I) pyruvate into 2-acetolactate;
II) 2-acetolactate into 2,3-dihydroxyisovaleriate;
III) 2,3-dihydroxyisovaleriate into 2-oxoisovaleriate;
IV) 2-oxoisovaleriate into isobutyraldehyde;
V) isobutyraldehyde into isobutanol; and
VI) isobutanol into isobutene
   and is described inter alia in WO 2011076689 and WO 2011076691.

According to a preferred embodiment of the invention the isobutene is derived in step a) from trisaccharides, disaccharides, monosaccharides, acetone or mixtures thereof. The tri- and disaccharides used are in particular raffinose, cellobiose, lactose, isomaltose, maltose and sucrose. The monosaccharides used are in particular D-glucose, D-fructose, D-galactose, D-mannose, DL-arabinose and DL-xylose. Herein the tri-, di- and monosaccharides inter alia originate (but not limited thereto)
from the digestion and the depolymerization of cellulose and hemicellulose using appropriate methods;
directly from plants with high sugar content such as sugar beet, sugar cane, palm sugar, maple sugar, sorghum, silver date palm, honey palm, palmyra palm and agaves by means of extraction;
from the depolymerization of plant starch by hydrolysis;
from the depolymerization of animal glycogen by hydrolysis;
directly from milk obtained from the dairy industry.

In a further preferred embodiment of the invention exclusively renewable raw materials are used for the fermentative production of isobutene. If desired, the origin of the carbon atoms derived from sources of renewable raw materials can be determined by the test method described in ASTM D6866. Herein the ratio of C¹⁴ to C¹² carbon isotopes is determined and compared with the isotopic ratio of a reference substance, the carbon atoms of which originate at 100% from sources of renewable raw materials. This test method is also known in modified form as radiocarbon method and is described among others in Olsson, I. U., 1991, Euro Courses: Advanced Scientific Techniques, volume 1, Issue Sci. Dating Methods, pages 15-35.

According to a preferred embodiment of the invention the fermentation process is carried out at temperatures of ≧20° C. to ≦45° C. and under atmospheric pressure, wherein isobutene is released as a gaseous product. This embodiment has the advantage that the thus obtained isobutene can be used again directly or after separation of inert gases.

Alternatively the fermentation process is carried out at temperatures of ≧20° C. to ≦45° C. and under a pressure between 1 to 30 bar in accordance with a likewise preferred embodiment of the invention. In this case, isobutene can be obtained as a liquid compound and be separated directly from the fermentation medium by phase separation. In this preferred embodiment the separation of inert gases can be considerably facilitated.

Depending in the embodiment step b) can preferably be carried out in two ways, and these likewise represent preferred embodiments of the present invention:
1. Conversion by a hydroformylation reaction/oxo reaction into isovaleraldehyde; and/or
2. Conversion in accordance with a Koch reaction into pivalic acid.

It is understood that the first way is especially chosen when isovaleraldehyde and its secondary products such as 3-methylbutanol and 3-methyl butyric acid are desired as reaction products, since isovaleraldehyde can be directly produced from isobutene.

In the following the two reaction options are discussed further:
1. Hydroformylation Reaction/Oxo Reaction

This reaction is carried out such that isovaleraldehyde preferably is derived from the reaction of isobutene with a synthesis gas preferably by use of cobalt or rhodium catalysts.

Rhodium or rhodium compounds can be used both as so-called “unmodified” catalysts, i.e. in the absence of complexing ligands, and in combination with complexing ligands, usually in combination with organophosphorous compounds, wherein the unmodified version especially is used when high n/iso ratios are of no interest and the formation of branched aldehydes is not possible and the olefinic substrate is relatively inert, respectively. The “unmodified” rhodium-catalysed hydroformylation requires with 20-30 MPa considerably more drastic reaction pressures than the “modified” methods, in which normally pressures of 1-10 MPa are used. Even slightly higher reaction temperatures may be necessary (Ullmann's Encyclopedia of Industrial Chemistry, Wiley-VCH, 6th edition, 2003, volume 24, pages 553-559).

One option of the modified method is the combination of rhodium compounds with water-soluble phosphines for use in two-phase controlled hydroformylation reactions, such as described in DE 2627354 or EP 0562451. Here, the catalyst and the ligand are present in the aqueous phase and the aldehyde produced forms an organic phase which can be separated from the aqueous catalyst solution in a simple way by means of phase separation.

The use of rhodium in combination with organophosphorus compounds can also be effected in a homogeneous phase. Here mainly triaryl- and trialkylphosphines such as triphenyl- and tricyclohexylphospin have been established which with respect to rhodium are used in an approximately 50-100 fold molar excess. Such complex compounds and their preparation are known (U.S. Pat. No. 3,527,809, U.S. Pat. No. 4,148,830, U.S. Pat. No. 4,247,486, U.S. Pat. No. 4,283,562).

In addition to phosphines depending on the application also phosphites (EP 0155508), bisphosphites (EP 0214622, DE 102009029050) and phosphacyclohexanes (U.S. Pat. No. 7,012,162) can be used as suitable ligands for rhodium-catalysed hydroformylations. These are characterized by generally significantly higher catalytic activities and significantly lower molar ratios of ligand-rhodium of ˜10. In addition, lower reaction pressures and temperatures can be used.

The rhodium compound and the ligand used may also be dissolved in an ionic liquid applied to a solid inert support material, liquid (SILP, supported ionic liquid phase) (DE 102010041821).
2. Koch Reaction

This reaction is preferably carried out such that isobutene in the presence of water and carbon monoxide under the influence of sulfuric acid, HF or H₃PO₄/BF₃ as a catalyst is transferred into pivalic acid (see Ullmann's Encyclopedia of Industrial Chemistry, Wiley-VCH, 6th edition, 2003, volume 6, page 503; Weissermel, Arpe, Industrielle Organische Chemie, VCH Verlagsgesellschaft, 3rd edition, 1988, pp. 150-152).

According to step c) optionally a further derivatisation can be carried out. Suitable derivatisations are described below, however, the invention is not limited thereto.

According to one embodiment of the invention, step c) includes an oxidation process. The conversion into 3-methylbutyric acid is preferably carried out by oxidation of the isovaleraldehyde in the presence of an oxygen-containing gas with absence or presence of a catalyst based on cerium, cobalt, chromium, copper, iron, manganese, molybdenum, nickel, vanadium or silver (Ullmann's Encyclopedia of Industrial Chemistry, Wiley-VCH, 6th edition, 2003, volume 6, pp. 497-98). The use of e.g. manganese acetate in combination with copper acetate is disclosed in U.S. Pat. No. 4,487,720. The oxidation can also be carried out in the presence of alkali and/or alkaline earth metal salts in combination with a metal or a compound of an element selected from groups 4-12, cerium or lanthanum (EP 1657230, US 20070265467).

The thus obtained 3-methylbutyric acid e.g. is a starting material for fungicides, rodenticides, (especially in the form of their ammonium salts), sedatives, anesthetics and other pharmaceuticals. The esters of 3-methylbutyric acid are used as lubricants, often as mixtures with other esterified aliphatic monocarboxylic acids, as solvents, plasticizers and in perfumes (Ullmann's Encyclopedia of Industrial Chemistry, Wiley-VCH, 6th edition, 2003, volume 6, pp. 500-502).

According to one embodiment of the invention, step c) comprises a reduction process. The reduction of isovaleraldehyde may take place depending on the application by means of hydrogenation in the gas or liquid phase at the metal contact. Preferred catalysts include nickel or copper catalysts.

Thus, according to a preferred embodiment of the invention the conversion of isovaleraldehyde into 3-methylbutanol under the influence of hydrogen-containing gas mixtures can take place at an elevated pressure at nickel-containing catalysts, as is described inter alia in DE 3932332 and DE 3932331. Hydrogenation catalysts and processes such as described in DE 102007041380 are likewise suitable for said reaction.

The thus obtained C₅-alcohol can, in turn, be converted into carboxylic acid esters. As such in DE 102006001795 dipentylterephthalic acid ester and in DE 102006026624 tripentylcitric acid ester are described which are suitable as fast gelling plasticizers for thermoplastics such as PVC.

According to one embodiment of the invention step c) includes a reductive amination process. By reaction with ammonia and hydrogen, the so-called reductive amination, isovaleraldehyde can be converted into the corresponding 3-methylbutylamines, wherein in addition to the primary also secondary and tertiary amine is formed (Ullmann's Encyclopedia of Industrial Chemistry, Wiley-VCH, 6th edition, 2003, volume 2, pp. 387-392). According to DE 10122758 mixed secondary amines can be produced under hydrogen pressure at a nickel-containing catalyst by the reaction of isovaleraldehyde with a primary amine or by reacting an aldehyde with 3-methylbutylamine. 3-Methylbutylamines can also be obtained by ammonolysis of 3-methylbutanol with ammonia, primary or secondary amines

According to one embodiment of the invention step c) includes an aldol reaction. In addition to the above described reactions into 3-methylbutanol, 3-methylbutyric acid and 3-methyl-butylamines branched decanols (EP 0562451) can be obtained by aldol condensation (e.g. U.S. Pat. No. 6,340,778, EP 603630) and complete hydrogenation and by partial hydrogenation of the adol condensation product followed by oxidation branched decanoic acids can be obtained. These products themselves in turn can be intermediates for the production of plasticizers, detergents and lubricants. By aldol reaction with acetone and partial hydrogenation of the product 6-methyl-2-heptanone can be obtained, which in turn is an intermediate for the production of fragrances, pharmaceuticals or feedstuff additives (WO 02072522).

According to one embodiment of the invention step c) comprises a reduction and subsequent dehydration. Another possibility for the transformation of isovaleraldehyde into valuable products is the reaction into 3-methyl-1-butene by dehydration of 3-methyl butanol described in DE 102006031964, which, as decribed above, can be obtained by hydrogenation of isovaleraldehyde. The olefin thus obtained can be used as a monomer or co-monomer for the production of polymers.

In a further embodiment of the invention step c) includes the reaction of isovaleraldehyde with formaldehyde and the subsequent hydrogenation of the methylenation product into 2,3-dimethylbutanol, which then is dehydrated into a mixture of 2,3-dimethyl-1-butene and 2,3-dimethyl-2-butene and isomerised into 2,3-dimethyl-2-butene. 2,3-dimethyl-2-butene is then transformed into pinacolone with hydrogen peroxide in the presence of a carboxylic acid (DE 2917779, EP 90246).

The pivalic acid already described can be further processed with alcohols into hardly saponifiable esters or by transvinylation with vinyl acetate or vinyl propionate into the vinyl ester of the pivalic acid, which is used as a comonomer for the production of dispersions which advantageously affect the hydrolysis resistance and moisture absorption of paints (Ullmanns Encyclopedia of Industrial Chemistry, Wiley-VCH, 6th edition, 2003 volume 38, pp. 70-73.)

According to a preferred embodiment of the invention between steps b) and c) no purification of the isopentane derivative is carried out because the isobutene resulting from step a) is so pure that no purification of the isopentane derivative has to be carried out. The term “purification” mutatis mutandis denotes the above methods.

Alternatively, in accordance with a likewise preferred embodiment between steps b) and c) a step b1) is carried out:
b1) purification of the isopentane derivative obtained in step b).

In the case of the isovaleraldehyde step b1) is preferably carried out by distillation; if pivalic acid is the reaction product, it can (because it is in the form a solid) also be purified by precipitation.

In some embodiments of the invention this has proved to be advantageous, since in this way the by-products formed in a small amount can be separated.

The synthesis steps to be used according to the invention, which are mentioned above and claimed and described in the embodiments do not underlie particular exceptional conditions with respect to their technical concept such that the selection criteria known in this field of application can be applied without restriction.

The individual combinations of components and features of the embodiments mentioned above are exemplary, the replacement and substitution of these teachings with other teachings that are included in this document with the documents cited are also explicitly contemplated. Those skilled in the art will recognize that variations, modifications and other embodiments different from those described herein may also occur without departing from the spirit and scope of the invention. Accordingly, the above description should be considered as exemplary and not as limiting. The word “comprise” used in the claims does not exclude other elements or steps. The indefinite article “a” does not exclude the meaning of a plural. The mere fact that certain amounts are recited in mutually different claims does not mean that a combination of these amounts can not be used to advantage. The scope of the invention is defined in the following claims and the associated equivalents.

1. Method of producing isopentane derivatives, comprising the steps of:
a) fermentative preparation of isobutene;
d) extension by one carbon atom in order to obtain an isopentane derivative; and
e) optionally further derivatisations.

2. Method according to claim 1, wherein between steps a) and b) no purification of the isobutene is carried out.

3. Method according to claim 1, wherein the isobutene in step a) is derived from trisaccharides, disaccharides, monosaccharides, acetone or mixtures thereof.

4. Method according to claim 1, wherein renewable raw materials are used for the fermentative production of isobutene.

5. Method according to claim 1, wherein the fermentation process is carried out at temperatures of ≧20° C. to ≦45° C. and under atmospheric pressure, and wherein isobutene is released as a gaseous product.

6. Method according to claim 1, wherein the fermentation process is carried out at temperatures of ≧20° C. to ≦45° C. and under a pressure between 1 to 30 bar.

7. Method according to claim 1, wherein step b) is carried out in accordance with a hydroformylation/oxo reaction.

8. Method according to claim 1, wherein step d) is carried out in accordance with a Koch reaction.

9. Method according to claim 1, wherein between steps b) and c) no purification of the isopentane derivative is carried out.

10. Method according to claim 1, wherein step c) comprises an oxidation, reduction, reductive amination, ammonolysis and/or aldol reaction.

11. Method according to claim 1, wherein 3-methylbutanal is produced as isopentane derivative.

12. Method according to claim 1, wherein 3-methylbutyric acid is produced as isopentane derivative.

13. Method according to claim 2, wherein renewable raw materials are used for the fermentative production of isobutene.

14. Method according to claim 2, wherein the fermentation process is carried out at temperatures of ≧20° C. to ≦45° C. and under atmospheric pressure, and wherein isobutene is released as a gaseous product.

15. Method according to claim 2, wherein the fermentation process is carried out at temperatures of ≧20° C. to ≦45° C. and under a pressure between 1 to 30 bar.

16. Method according to claim 2, wherein step b) is carried out in accordance with a hydroformylation/oxo reaction.

17. Method according to claim 2, wherein step d) is carried out in accordance with a Koch reaction.

18. Method according to claim 2, wherein step c) comprises an oxidation, reduction, reductive amination, ammonolysis and/or aldol reaction.

19. Method according to claim 2, wherein 3-methylbutanal is produced as isopentane derivative.

20. Method according to claim 2, wherein 3-methylbutyric acid is produced as isopentane derivative.
